(19) United States
(12) Patent Application Publication
(10) Pub. No.: US 2016/0067247 A1
(43) Pub. Date: Mar. 10, 2016

(54) NOVEL USE

(71) Applicant: GlaxoSmithKline Intellectual Property (No.2) Limited, Brentford (GB)

(72) Inventors: Richard Francis WOOSTER, Collegeville, PA (US); Pauline Teresa Lukey, Stevenage (GB); Patrick John Thompson Vallance, Brentford (GB)

(21) Appl. No.: 14/944,531

(22) Filed: Nov. 18, 2015

Related U.S. Application Data
(63) Continuation of application No. 14/376,664, filed on Aug. 5, 2014.
(60) Provisional application No. 61/702,854, filed on Sep. 19, 2012.
(60) Provisional application No. 61/595,293, filed on Feb. 6, 2012.

(51) Int. Cl. A61K 31/501

(52) U.S. Cl. CPC A61K 31/501

(57) ABSTRACT

The present invention is directed to compounds or pharmaceutically acceptable salts thereof for use in the treatment of fibrotic diseases such as idiopathic pulmonary fibrosis (IPF).

### FIELD OF THE INVENTION

The present invention is directed to compounds and pharmaceutically acceptable salts thereof which are inhibitors of the activity or function of the phosphoinositide 3′OH kinase family (hereinafter PI3K), which includes PI3Kα, PI3Kβ, PI3Kγ and PI3Kδ, and the mammalian target of rapamycin (hereinafter mTOR), a PI3K downstream signalling target, for use in the treatment of fibrotic diseases, in particular idiopathic pulmonary fibrosis (hereinafter IPF).

### BACKGROUND OF THE INVENTION

Fibrotic diseases involve the formation of excess fibrous connective tissue in an organ or tissue in a reparative or reactive process. Such fibrotic diseases include IPF, pulmonary fibrosis, interstitial lung diseases, non-specific interstitial pneumonia (NSIP), usual interstitial pneumonia (UIP), endomyocardial fibrosis, mediastinal fibrosis, myelofibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, and fibro-proliferative disorders.

IPF represents the end-stage of a heterogeneous group of acute and chronic respiratory disorders where patient prognosis is poor, with a typical survival time of less than 5 years. The cardinal lesions of IPF are fibrotic foci, consisting of reactive and hyperplastic epithelial cells overlaying a dense core of hyperproliferative fibroblasts and myofibroblasts which appear to be resistant to apoptosis and deposit excessive amounts of extracellular matrix proteins within the pulmonary interstitium, leading to airspace obliteration and respiratory insufficiency.

The Class I PI3 kinases catalyse the conversion of Ptdln(4,5)P2 to Ptdln(3,4,5)P3, inducing recruitment and phosphorylation of downstream signalling kinase (most notably AKT) to the plasma membrane and resulting in the activation of multiple signalling cascades involved in essential cellular functions including cell proliferation, metabolism, growth, and survival.

The Class I PI3 kinase family comprises 4 separate isoforms (α,β,γ,δ) distinguished by the sequence and structure of the p110 catalytic subunit. Of the four isoforms, α and β are ubiquitously expressed whereas γ and δ are enriched in leukocytes. Embryonic lethality is observed in mice homogenously expressing kinase dead α and β isoforms while heterogenous kinase dead mice exhibit partial lethality with metabolic and vascular defects. In-line with expression patterns, ablation of γ and δ isoforms results in immunological defects. PI3 kinase activity is negatively regulated by SHIP phosphatases, most notably in the case of Class I PI3 Kinase by PTEN, and it is well established that oncogenicity in a variety of tumour settings is promoted by dysreulated PI3K signalling resulting from either p110 mutation (p110α), over-expression (p110β, γ, δ) or alternatively by reduced functionality of the regulatory phosphatises.¹

Fibroblast survival, proliferation and matrix synthesis are central to the pathology of fibrosis, and it is likely that aberrant PI3 kinase signalling may play a critical role in both disease initiation and progression impacting on each of these fibroblast functions. In support of this, PI3 kinase has been implicated in collagen production and proliferation in lung fibroblasts² moreover dysregulated PI3-kinase signalling, resulting from a functional PTEN deficit, is associated with a hyper-proliferative phenotype in primary lung fibroblasts isolated from IPF patients.^{3,4} Additionally, mice deficient in PTEN show increased fibrosis following bleomycin induced lung injury.³ In the setting of dermal fibrosis, fibroblasts isolated from patients with systemic sclerosis show decreased PTEN expression associated with augmented pAKT signalling, while dermal fibroblasts isolated from PTEN conditional knockout mice exhibit PI3K dependant over-expression of collagen 1, α-SMA and in addition to the pro-fibrotic mediator CTGF.⁵ In a rat model of hepatic fibrosis PTEN expression is also down regulated, conferring a profibrotic phenotype.⁶ PI3 kinase signalling downstream of TGFβ is implicated in the differentiation of primary human lung fibroblasts into myofibroblasts⁷ and is found to convey resistance to apoptosis.⁸ Moreover the antifibrotic mediator PGE2, which is reduced in IPF lungs, enhances fibroblast apoptosis by inhibition of the AKT signalling pathway.⁹

It has been postulated that occult viral infections may act as cofactors in the pathogenesis of pulmonary fibrosis, either chronically by inducing genetic genetic instability and dysfunctional repair mechanisms, or acutely by triggering virally induced exacerbations.¹⁰ Activation of PI3K-Akt signaling is a strategy employed by certain viruses (for example adenovirus and influenza A) to facilitate viral penetration, slow down apoptosis or prolong viral replication in both acute and persistent infection.¹¹

There remains a need to provide compounds which are inhibitors of the activity or function of PI3K and mTOR which may be useful in the treatment of fibrotic diseases such as IPF.

### REFERENCES

1. Vanhaesebroeck, B., J. Guillermet-Guibert, M. Graupera, and B. Bilanges. 2010. The emerging mechanisms of isoform-specific PI3K signalling. Nat. Rev. Mol. Cell Biol. 11:329-341
2. Lu, Y., N. Azad, L. Wang, A. K. Iyer, V. Castranova, B. H. Jiang, and Y. Rojanasakul. 2010. Phosphatidylinositol-3-kinase/akt regulates bleomycin-induced fibroblast proliferation and collagen production. Am. J. Respir. Cell Mol. Biol. 42:432-441.
3. Xia, H., D. Diebold, R. Nho, D. Perlman, J. Kleidon, J. Kahm, S. Avdulov, M. Peterson, J. Nerva, P. Bitterman, et al. 2008. Pathological integrin signaling enhances proliferation of primary lung fibroblasts from patients with idiopathic pulmonary fibrosis. J. Exp. Med. 205:1659-1672.
4. Xia, H., W. Khalil, J. Kahm, J. Jessurun, J. Kleidon, and C. A. Henke. 2010. Pathologic caveolin-1 regulation of PTEN in idiopathic pulmonary fibrosis. Am. J. Pathol. 176:2626-2637.
5. Parapuram, S. K., X. Shi-Wen, C. Elliott, I. D. Welch, H. Jones, M. Baron, C. P. Denton, D. J. Abraham, and A. Leask. 2011. Loss of PTEN Expression by Dermal Fibroblasts Causes Skin Fibrosis. J. Invest Dermatol.
6. Hao, L. S., X. L. Zhang, J. Y. An, J. Karlin, X. P. Tian, Z. N. Dun, S. R. Xie, and S. Chen. 2009. PTEN expression is down-regulated in liver tissues of rats with hepatic fibrosis induced by biliary stenosis. APMIS 117:681-691.
7. Kulkarni, A. A., T. H. Thatcher, K. C. Olsen, S. B. Maggirwar, R. P. Phipps, and P. J. Sime. 2011. PPAR-gamma ligands repress TGFbeta-induced myofibroblast differentiation by targeting the PI3K/Akt pathway: implications for therapy of fibrosis. PLoS. One. 6:e15909.
8. Horowitz, J. C., D. Y. Lee, M. Waghray, V. G. Keshamouni, P. E. Thomas, H. Zhang, Z. Cui, and V. J. Thannickal. 2004. Activation of the pro-survival phosphatidylinositol 3-kinase/AKT pathway by transforming growth factor-beta1 in mesenchymal cells is mediated by p38 MAPK-dependent induction of an autocrine growth factor. J. Biol. Chem. 279:1359-1367.
9. Maher, T. M., I. C. Evans, S. E. Bottoms, P. F. Mercer, A. J. Thorley, A. G. Nicholson, G. J. Laurent, T. D. Tetley, R. C. Chambers, and R. J. McAnulty. 2010. Diminished prostaglandin E2 contributes to the apoptosis paradox in idiopathic pulmonary fibrosis. Am. J. Respir. Crit Care Med. 182:73-82.
10. Naik P K, Moore B B. 2010. Viral infection and aging as cofactors for the development of pulmonary fibrosis. Expert Rev. Respir. Med. 4: 759-71.
11. Ji W T, Liu H J. 2008. PI3K-Akt signaling and viral infection. Recent Pat. Biotechnol. 2: 218-26.

### SUMMARY OF THE INVENTION

The present invention provides a compound of formula (I)

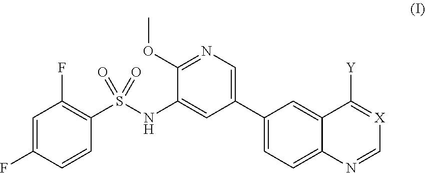

wherein
X is —CH— and Y is 4-pyridazinyl; or
X is —N— and Y is 4-morpholinyl;
or a pharmaceutically acceptable salt thereof for use in the treatment of a fibrotic disease.

### BRIEF DESCRIPTION OF THE FIGURES

FIG. 1 shows a schematic of the experimental regimen for priming with TGFβ for 24 hours and TGFβ subsequently removed.

FIG. 2 shows a schematic of the experimental regimen for priming with TGFβ for 24 hours followed by continual TGFβ stimulation.

FIG. 3 shows the percentage of AKT phosphorylated at Ser473 after 2,4-difluoro-N-{2-(methyloxy)-5-[4-(4-pyridazinyl)-6-quinolinyl]-3-pyridinyl}benzenesulfonamide treatment. Representative concentration response to 2,4-difluoro-N-{2-(methyloxy)-5-[4-(4-pyridazinyl)-6-quinolinyl]-3-pyridinyl}benzenesulfonamide of non-IPF (A) and IPF (B) fibroblast lines. Data normalized to AKT phosphorylation of FBS treated cells alone (100%). Data shown as mean+/−SEM of n=3 replicate wells per data point. Curves were fitted using a 4 parameter non-linear regression. Individual cell lines and passage numbers are listed in legend.

FIG. 4 shows the effect of 2,4-difluoro-N-{2-(methyloxy)-5-[4-(4-pyridazinyl)-6-quinolinyl]-3-pyridinyl}benzenesulfonamide on cell proliferation of fibroblasts after FBS treatment. Representative dose response to 2,4-difluoro-N-{2-(methyloxy)-5-[4-(4-pyridazinyl)-6-quinolinyl]-3-pyridinyl}benzenesulfonamide of non-IPF (A) and IPF (B) cell lines. Data normalized to % of maximal FBS response (% upper asymptote). Data shown as mean+/−SEM of n=5 or 6 replicate wells per data point. Dashed line is the mean signal for cells in serum free medium containing 0.1% DMSO for each cell line. Curves were fitted using a 4 parameter non-linear regression. Individual cell lines and passage numbers are listed in legend.

FIG. 5 shows the effect of 2,4-difluoro-N-{2-(methyloxy)-5-[4-(4-pyridazinyl)-6-quinolinyl]-3-pyridinyl}benzenesulfonamide on pro-collagen accumulation in the supernatants of TGFβ differentiated myofibroblasts. The effect of 2,4-difluoro-N-{2-(methyloxy)-5-[4-(4-pyridazinyl)-6-quinolinyl]-3-pyridinyl}benzenesulfonamide on TGFβ-induced procollagen synthesis in vitro by non-IPF fibroblast lines: Fibroblast lines 0110 (A) and 0610 (B) were incubated with TGFβ (1 ng/ml) for 24 hours. TGFβ containing medium was subsequently removed and replaced with fresh medium containing 3 nM or 30 nM 2,4-difluoro-N-{2-(methyloxy)-5-[4-(4-pyridazinyl)-6-quinolinyl]-3-pyridinyl}benzenesulfonamide. The cells were incubated with the compound for further 24 and 48 hours. Data are expressed as mean±S.E.M. for 3 replicates and normalised to cell number. The values *p<0.05, **p<0.1 and ***p<0.001 denote statistical significance (TWO-WAY ANOVA, Bonferroni analysis) of the indicated data compared to cells treated with TGFβ alone.

FIG. 6 shows the effect of 2,4-difluoro-N-{2-(methyloxy)-5-[4-(4-pyridazinyl)-6-quinolinyl]-3-pyridinyl}benzenesulfonamide on pro-collagen accumulation in the supernatants of TGFβ differentiated myofibroblasts following replenishment of TGFβ. The effect of 2,4-difluoro-N-{2-(methyloxy)-5-[4-(4-pyridazinyl)-6-quinolinyl]-3-pyridinyl}benzenesulfonamide on TGFβ-induced pro-collagen synthesis in vitro by non-IPF fibroblast lines: Fibroblast lines 0110 (A) and 0610 (B) were incubated with TGFβ (1 ng/ml) for 24 hours. TGFβ was subsequently removed and replaced with fresh medium containing TGFβ and 3 nM or 30 nM 2,4-difluoro-N-{2-(methyloxy)-5-[4-(4-pyridazinyl)-6-quinolinyl]-3-pyridinyl}benzenesulfonamide. The cells were incubated with the compound for further 24 and 48 hours. Data are expressed as mean±S.E.M. for 3 replicates and normalised to cell number. The values **p<0.01 and ***p<0.001 denote statistical significance (TWO-WAY ANOVA, Bonferroni analysis) of the indicated data compared to cells treated with TGFβ alone.

### DETAILED DESCRIPTION OF THE INVENTION

In one embodiment, the present invention provides a compound of formula (I)

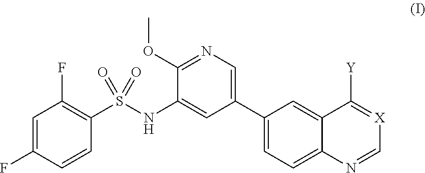

wherein
X is —CH— and Y is 4-pyridazinyl; or
X is —N— and Y is 4-morpholinyl;
or a pharmaceutically acceptable salt thereof for use in the treatment of a fibrotic disease.

In another embodiment, the present invention provides a compound which is 2,4-difluoro-N-{2-(methyloxy)-5-[4-(4-pyridazinyl)-6-quinolinyl]-3-pyridinyl}benzenesulfonamide:

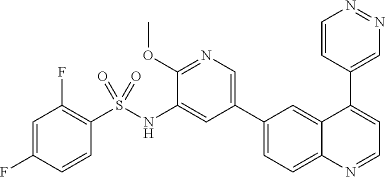 [SMILES: COc1ncc(-c2ccc3nccc(-c4ccnnc4)c3c2)cc1NS(=O)(=O)c1ccc(F)cc1F]

or a pharmaceutically acceptable salt thereof for use in the treatment of a fibrotic disease.

In another embodiment, the present invention provides a compound which is 2,4-difluoro-N-{2-(methyloxy)-5-[4-(4-pyridazinyl)-6-quinolinyl]-3-pyridinyl}benzenesulfonamide:

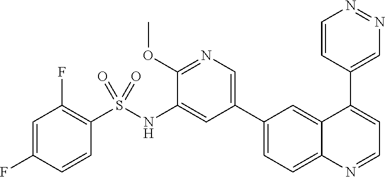 [SMILES: COc1ncc(-c2ccc3nccc(-c4ccnnc4)c3c2)cc1NS(=O)(=O)c1ccc(F)cc1F]

for use in the treatment of a fibrotic disease.

In another embodiment, the present invention provides a compound which is 2,4-difluoro-N-{2-(methyloxy)-5-[4-(4-pyridazinyl)-6-quinolinyl]-3-pyridinyl}benzenesulfonamide:

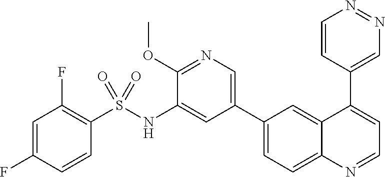 [SMILES: COc1ncc(-c2ccc3nccc(-c4ccnnc4)c3c2)cc1NS(=O)(=O)c1ccc(F)cc1F]

or a pharmaceutically acceptable salt thereof for use in the treatment of IPF.

In a further embodiment, the present invention provides a compound which is 2,4-difluoro-N-{2-(methyloxy)-5-[4-(4-pyridazinyl)-6-quinolinyl]-3-pyridinyl}benzenesulfonamide:

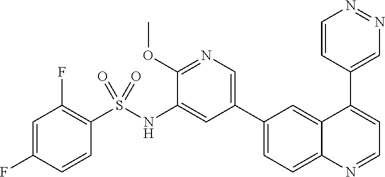 [SMILES: COc1ncc(-c2ccc3nccc(-c4ccnnc4)c3c2)cc1NS(=O)(=O)c1ccc(F)cc1F]

for use in the treatment of IPF.

Included within the scope of the invention is the use of all solvates (including hydrates), complexes, polymorphs, prodrugs and radiolabelled derivatives of a compound of formula (I) or a pharmaceutically acceptable salt thereof.

Compounds of formula (I) may be administered as a pharmaceutically acceptable salt. As used herein, the term “pharmaceutically acceptable salt” refers to a salt that retains the desired biological activity of the compound and exhibits minimal undesired toxicological effects. Pharmaceutically acceptable salts of compounds may be used to impart greater stability or solubility to a molecule thereby facilitating formulation into a dosage form. These pharmaceutically acceptable salts may be prepared in situ during the final isolation and purification of the compound, or by separately reacting the purified compound, or a non-pharmaceutically acceptable salt thereof, with a suitable base or acid. For a review on suitable salts see Berge et al., J. Pharm. Sci., 1977, 66, 1-19. In one embodiment, the invention provides the use of a pharmaceutically acceptable salt of 2,4-difluoro-N-{2-(methyloxy)-5-[4-(4-pyridazinyl)-6-quinolinyl]-3-pyridinyl}benzenesulfonamide. In a further embodiment, the invention provides the use of 2,4-difluoro-N-{2-(methyloxy)-5-[4-(4-pyridazinyl)-6-quinolinyl]-3-pyridinyl}benzenesulfonamide as the free base.

### Compound Preparation

The compounds for use according to the invention may be made by a variety of methods, including standard chemistry. For example, 2,4-difluoro-N-{2-(methyloxy)-5-[4-(4-pyridazinyl)-6-quinolinyl]-3-pyridinyl}benzenesulfonamide may be prepared as described in WO 2008/144463 and 2,4-difluoro-N-{2-(methyloxy)-5-[4-(4-morpholinyl)-6-quinazolinyl]-3-pyridinyl}benzenesulfonamide may be prepared as described in WO 2008/157191.

### Methods of Use

The methods of treatment of the invention comprise administering a safe and effective amount of a compound of formula (I) or a pharmaceutically acceptable salt thereof to a patient in need thereof.

As used herein, “treat” in reference to a disorder means: (1) to ameliorate the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As used herein, “safe and effective amount” in reference to a compound of formula (I) or a pharmaceutically acceptable salt thereof, or other pharmaceutically-active agent, means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human (including adults and children) or other animal. In one embodiment, “patient” refers to a human.

The compound or a pharmaceutically acceptable salt thereof may be administered by any suitable route of administration, in particular oral administration.

The compound or a pharmaceutically acceptable salt thereof may be administered according to a dosing regimen wherein a number of doses are administered at varying intervals of time for a given period of time. For example, doses may be administered one, two, three, or four times per day. In one embodiment, a dose is administered twice per day (BID).

Doses may be administered until the desired therapeutic effect is achieved or indefinitely to maintain the desired therapeutic effect. Suitable dosing regimens, including the duration such regimens are administered, may depend on the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages for oral administration may range from about 0.1 mg to about 20 mg, for example from about 0.1 mg to about 10 mg such as about 0.4 mg to about 7 mg. For example, a dose of from about 0.1 mg to about 5 mg, for example from about 0.2 mg to about 3.5 mg such as from about 0.25 mg to about 3 mg, may be administered BID per patient. In one embodiment, a dose of from about 0.25 mg to about 2.5 mg may be administered BID per patient.

In one aspect, the invention provides a compound of formula (I) or a pharmaceutically acceptable salt thereof for use in the treatment of a fibrotic disease.

Such fibrotic diseases may include IPF, pulmonary fibrosis, interstitial lung diseases, non-specific interstitial pneumonia (NSIP), usual interstitial pneumonia (UIP), endomyocardial fibrosis, mediastinal fibrosis, myelofibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, and fibro-proliferative disorders. In one embodiment, fibrotic diseases include IPF, pulmonary fibrosis, interstitial lung diseases, non-specific interstitial pneumonia (NSIP), usual interstitial pneumonia (UIP), endomyocardial fibrosis, mediastinal fibrosis, myelofibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, hypertrophic cardiomyopathy (HCM), hypertension-related nephropathy, radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, or fibro-proliferative disorders. In a further embodiment, the fibrotic disease is IPF.

In one embodiment, the invention provides the use of a compound of formula (I) or a pharmaceutically acceptable salt thereof in the manufacture of a medicament for use in the treatment of a fibrotic disease.

In a further embodiment, the invention provides a method of treating a fibrotic disease comprising administering a safe and effective amount of a compound of formula (I) or a pharmaceutically acceptable salt thereof to a patient in need thereof.

In another aspect, the invention provides a compound which is 2,4-difluoro-N-{2-(methyloxy)-5-[4-(4-pyridazinyl)-6-quinolinyl]-3-pyridinyl}benzenesulfonamide or a pharmaceutically acceptable salt thereof for use in the treatment of a fibrotic disease.

In one embodiment, the invention provides the use of a compound which is 2,4-difluoro-N-{2-(methyloxy)-5-[4-(4-pyridazinyl)-6-quinolinyl]-3-pyridinyl}benzenesulfonamide or a pharmaceutically acceptable salt thereof in the manufacture of a medicament for use in the treatment of a fibrotic disease.

In a further embodiment, the invention provides a method of treating a fibrotic disease comprising administering a safe and effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof to a patient in need thereof.

In another aspect, the invention provides a compound which is 2,4-difluoro-N-{2-(methyloxy)-5-[4-(4-pyridazinyl)-6-quinolinyl]-3-pyridinyl}benzenesulfonamide for use in the treatment of a fibrotic disease.

In one embodiment, the invention provides the use of a compound which is 2,4-difluoro-N-{2-(methyloxy)-5-[4-(4-pyridazinyl)-6-quinolinyl]-3-pyridinyl}benzenesulfonamide in the manufacture of a medicament for use in the treatment of a fibrotic disease.

In a further embodiment, the invention provides a method of treating a fibrotic disease comprising administering a safe and effective amount of 2,4-difluoro-N-{2-(methyloxy)-5-[4-(4-pyridazinyl)-6-quinolinyl]-3-pyridinyl}benzenesulfonamide to a patient in need thereof.

In a further aspect, the invention provides a compound of formula (I) or a pharmaceutically acceptable salt thereof for use in the treatment of IPF.

In one embodiment, the invention provides the use of a compound of formula (I) or a pharmaceutically acceptable salt thereof in the manufacture of a medicament for use in the treatment of IPF.

In a further embodiment, the invention provides a method of treating IPF comprising administering a safe and effective amount of a compound of formula (I) or a pharmaceutically acceptable salt thereof to a patient in need thereof.

In another aspect, the invention provides a compound which is 2,4-difluoro-N-{2-(methyloxy)-5-[4-(4-pyridazinyl)-6-quinolinyl]-3-pyridinyl}benzenesulfonamide or a pharmaceutically acceptable salt thereof for use in the treatment of IPF.

In one embodiment, the invention provides the use of a compound which is 2,4-difluoro-N-{2-(methyloxy)-5-[4-(4-pyridazinyl)-6-quinolinyl]-3-pyridinyl}benzenesulfonamide or a pharmaceutically acceptable salt thereof in the manufacture of a medicament for use in the treatment of IPF.

In a further embodiment, the invention provides a method of treating IPF comprising administering a safe and effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof to a patient in need thereof.

In a further aspect, the invention provides a compound which is 2,4-difluoro-N-{2-(methyloxy)-5-[4-(4-pyridazinyl)-6-quinolinyl]-3-pyridinyl}benzenesulfonamide for use in the treatment of IPF.

In one embodiment, the invention provides the use of a compound which is 2,4-difluoro-N-{2-(methyloxy)-5-[4-(4-pyridazinyl)-6-quinolinyl]-3-pyridinyl}benzenesulfonamide in the manufacture of a medicament for use in the treatment of IPF.

In a further embodiment, the invention provides a method of treating IPF comprising administering a safe and effective amount of 2,4-difluoro-N-{2-(methyloxy)-5-[4-(4-pyridazinyl)-6-quinolinyl]-3-pyridinyl}benzenesulfonamide to a patient in need thereof.

### Compositions

Compounds of formula (I) or pharmaceutically acceptable salts thereof may be formulated into a pharmaceutical composition prior to administration to a patient.

Accordingly, in one aspect the invention is directed to pharmaceutical compositions comprising a compound of formula (I) or a pharmaceutically acceptable salt thereof, and one or more pharmaceutically acceptable excipients for use in the treatment of a fibrotic disease.

In one embodiment, the invention is directed to pharmaceutical compositions comprising compound of formula (I) or a pharmaceutically acceptable salt thereof, and one or more pharmaceutically acceptable excipients for use in the treatment of IPF.

In another embodiment, the invention is directed to pharmaceutical compositions comprising 2,4-difluoro-N-{2-(methyloxy)-5-[4-(4-pyridazinyl)-6-quinolinyl]-3-pyridinyl}benzenesulfonamide or a pharmaceutically acceptable salt thereof, and one or more pharmaceutically acceptable excipients for use in the treatment of a fibrotic disease.

In another embodiment, the invention is directed to pharmaceutical compositions comprising 2,4-difluoro-N-{2-(methyloxy)-5-[4-(4-pyridazinyl)-6-quinolinyl]-3-pyridinyl}benzenesulfonamide, and one or more pharmaceutically acceptable excipients for use in the treatment of a fibrotic disease.

In another embodiment, the invention is directed to pharmaceutical compositions comprising 2,4-difluoro-N-{2-(methyloxy)-5-[4-(4-pyridazinyl)-6-quinolinyl]-3-pyridinyl}benzenesulfonamide or a pharmaceutically acceptable salt thereof, and one or more pharmaceutically acceptable excipients for use in the treatment of IPF.

In a further embodiment, the invention is directed to pharmaceutical compositions comprising 2,4-difluoro-N-{2-(methyloxy)-5-[4-(4-pyridazinyl)-6-quinolinyl]-3-pyridinyl}benzenesulfonamide, and one or more pharmaceutically acceptable excipients for use in the treatment of IPF.

In another aspect the invention is directed to pharmaceutical compositions comprising 0.1 mg to about 5 mg of a compound of formula (I) or a pharmaceutically acceptable salt thereof and about 0.1 g to about 2 g of one or more pharmaceutically acceptable excipients for use in the treatment of a fibrotic disease.

In one embodiment, the invention is directed to pharmaceutical compositions comprising 0.1 mg to about 5 mg of a compound of formula (I) or a pharmaceutically acceptable salt thereof and about 0.1 g to about 2 g of one or more pharmaceutically acceptable excipients for use in the treatment of IPF.

In another embodiment, the invention is directed to pharmaceutical compositions comprising 0.1 mg to about 5 mg of 2,4-difluoro-N-{2-(methyloxy)-5-[4-(4-pyridazinyl)-6-quinolinyl]-3-pyridinyl}benzenesulfonamide or a pharmaceutically acceptable salt thereof and about 0.1 g to about 2 g of one or more pharmaceutically acceptable excipients for use in the treatment of a fibrotic disease.

In another embodiment, the invention is directed to pharmaceutical compositions comprising 0.1 mg to about 5 mg of 2,4-difluoro-N-{2-(methyloxy)-5-[4-(4-pyridazinyl)-6-quinolinyl]-3-pyridinyl}benzenesulfonamide or a pharmaceutically acceptable salt thereof and about 0.1 g to about 2 g of one or more pharmaceutically acceptable excipients for use in the treatment of IPF.

In another embodiment, the invention is directed to pharmaceutical compositions comprising 0.1 mg to about 5 mg of 2,4-difluoro-N-{2-(methyloxy)-5-[4-(4-pyridazinyl)-6-quinolinyl]-3-pyridinyl}benzenesulfonamide or a pharmaceutically acceptable salt thereof and about 0.1 g to about 2 g of one or more pharmaceutically acceptable excipients for use in the treatment of a fibrotic disease.

In a further embodiment, the invention is directed to pharmaceutical compositions comprising 0.1 mg to about 5 mg of 2,4-difluoro-N-{2-(methyloxy)-5-[4-(4-pyridazinyl)-6-quinolinyl]-3-pyridinyl}benzenesulfonamide and about 0.1 g to about 2 g of one or more pharmaceutically acceptable excipients for use in the treatment of IPF.

In a further aspect the invention is directed to a pharmaceutical composition for the treatment of a fibrotic disease comprising a compound of formula (I) or a pharmaceutically acceptable salt thereof.

In one embodiment, the invention is directed to a pharmaceutical composition for the treatment of a fibrotic disease comprising 2,4-difluoro-N-{2-(methyloxy)-5-[4-(4-pyridazinyl)-6-quinolinyl]-3-pyridinyl}benzenesulfonamide or a pharmaceutically acceptable salt thereof.

In another embodiment, the invention is directed to a pharmaceutical composition for the treatment of a fibrotic disease comprising 2,4-difluoro-N-{2-(methyloxy)-5-[4-(4-pyridazinyl)-6-quinolinyl]-3-pyridinyl}benzenesulfonamide.

In another embodiment, the invention is directed to a pharmaceutical composition for the treatment of IPF comprising a compound of formula (I) or a pharmaceutically acceptable salt thereof.

In another embodiment, the invention is directed to a pharmaceutical composition for the treatment of IPF comprising 2,4-difluoro-N-{2-(methyloxy)-5-[4-(4-pyridazinyl)-6-quinolinyl]-3-pyridinyl}benzenesulfonamide or a pharmaceutically acceptable salt thereof.

In further embodiment, the invention is directed to a pharmaceutical composition for the treatment of IPF comprising 2,4-difluoro-N-{2-(methyloxy)-5-[4-(4-pyridazinyl)-6-quinolinyl]-3-pyridinyl}benzenesulfonamide.

The pharmaceutical compositions for use according to the invention may be prepared and packaged in bulk form wherein a safe and effective amount of a compound of formula (I) or a pharmaceutically acceptable salt thereof can be extracted and then given to the patient such as with powders or syrups. Alternatively, the pharmaceutical compositions for use according to the invention may be prepared and packaged in unit dosage form wherein each physically discrete unit contains a compound of formula (I) or a pharmaceutically acceptable salt thereof. When prepared in unit dosage form, the pharmaceutical compositions for use according to the invention typically may contain, for example, from about 0.1 mg to about 5 mg, for example from about 0.2 mg to about 3.5 mg such as from about 0.25 mg to about 3 mg of a compound of formula (I) or a pharmaceutically acceptable salt thereof. In one embodiment, the pharmaceutical compositions for use according to the invention typically contain about 0.25 mg of a compound of formula (I) or a pharmaceutically acceptable salt thereof. In a further embodiment, the pharmaceutical compositions for use according to the invention typically contain about 0.5 mg of a compound of formula (I) or a pharmaceutically acceptable salt thereof.

As used herein, “pharmaceutically acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of a compound of formula (I) or a pharmaceutically acceptable salt thereof when administered to a patient and interactions which would result in pharmaceutical compositions that are not pharmaceutically acceptable are avoided. In addition, each excipient must of course be pharmaceutically-acceptable eg of sufficiently high purity.

The compounds of formula (I) or pharmaceutically acceptable salts thereof and the pharmaceutically acceptable excipient or excipients will typically be formulated into a dosage form adapted for administration to the patient by the desired route of administration. For example, dosage forms include those adapted for oral administration such as tablets, capsules, caplets, pills, troches, powders, syrups, elixers, suspensions, solutions, emulsions, sachets, and cachets.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of a compound of formula (I) or a pharmaceutically acceptable salt thereof once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other excipients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions for use according to the invention are prepared using techniques and methods known to those skilled in the art. Some of the methods commonly used in the art are described in Remington's Pharmaceutical Sciences (Mack Publishing Company).

A pharmaceutical composition comprising a compound of formula (I) or a pharmaceutically acceptable salt thereof may be prepared by, for example, admixture at ambient temperature and atmospheric pressure.

In one embodiment, a compound of formula (I) or a pharmaceutically acceptable salt thereof will be formulated for oral administration. For example, the composition for use according to the invention may be a solid oral dosage form such as a tablet or capsule comprising a safe and effective amount of a compound of formula (I) or a pharmaceutically acceptable salt thereof and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesuim stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compound of formula (I) or a pharmaceutically acceptable salt thereof may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compound of formula (I) or a pharmaceutically acceptable salt thereof may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In another aspect, the composition for use according to the invention is a liquid oral dosage form. Oral liquids such as solution, syrups and elixirs can be prepared in dosage unit form so that a given quantity contains a predetermined amount of a compound of formula (I) or a pharmaceutically acceptable salt thereof. Syrups can be prepared by dissolving a compound of formula (I) or a pharmaceutically acceptable salt thereof in a suitably flavored aqueous solution, while elixirs are prepared through the use of a non-toxic alcoholic vehicle. Suspensions can be formulated by dispersing a compound of formula (I) or a pharmaceutically acceptable salt thereof in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

According to the invention, a compound of formula (I) or a pharmaceutically acceptable salt thereof may be used in combination with one or more other therapeutic agents, in the treatment of a fibrotic disease.

Suitable therapeutic agents for use in combination with a compound of formula (I) or a pharmaceutically acceptable salt thereof include anti-inflammatory agents (for example corticosteroids such as prednisone), immunosuppressants (for example azathioprine or cyclophosphamide), anti-proliferatives, pirfenidone, N-acetylcysteine, p38 MAK kinase inhibitors (for example losmapimod, (6-[5-(cyclopropylcarbamoyl)-3-fluoro-2-methylphenyl]-N-(2,2-dimethylpropyl)pyridine-3-carboxamide) and MEK or dual MEK1/MEK2 inhibitors (for example selumetinib, 5-(4-bromo-2-chlorophenylamino)-4-fluoro-N-(2-hydroxyethoxy)-1-methyl-1H-benzo[d]imidazole-6-carboxamide).

The invention thus provides, in one aspect, a combination comprising a compound of formula (I) or a pharmaceutically acceptable salt thereof and one or more other therapeutically active agents for use in the treatment of a fibrotic disease.

In one embodiment, the invention provides a method of treating a fibrotic disease comprising administering a safe and effective amount of a combination comprising a compound of formula (I) or a pharmaceutically acceptable salt thereof and one or more therapeutically active agents.

In a further embodiment, the invention provides a combination comprising a compound of formula (I) or a pharmaceutically acceptable salt thereof and one or more therapeutically active agents in the manufacture of a medicament for use in the treatment of a fibrotic disease.

In another aspect, the invention provides a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more other therapeutically active agents for use in the treatment of a fibrotic disease.

In one embodiment, the invention provides a method of treating a fibrotic disease comprising administering a safe and effective amount of a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more therapeutically active agents.

In a further embodiment, the invention provides a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more therapeutically active agents in the manufacture of a medicament for use in the treatment of a fibrotic disease.

In another aspect, the invention provides a combination comprising 2,4-difluoro-N-{2-(methyloxy)-5-[4-(4-pyridazinyl)-6-quinolinyl]-3-pyridinyl}benzenesulfonamide and one or more other therapeutically active agents for use in the treatment of a fibrotic disease.

In another embodiment, the invention provides a method of treating a fibrotic disease comprising administering a safe and effective amount of a combination comprising 2,4-difluoro-N-{2-(methyloxy)-5-[4-(4-pyridazinyl)-6-quinolinyl]-3-pyridinyl}benzenesulfonamide and one or more therapeutically active agents.

In a further embodiment, the invention provides a combination comprising 2,4-difluoro-N-{2-(methyloxy)-5-[4-(4-pyridazinyl)-6-quinolinyl]-3-pyridinyl}benzenesulfonamide and one or more therapeutically active agents in the manufacture of a medicament for use in the treatment of a fibrotic disease.

In another aspect, the invention provides a combination comprising a compound of formula (I) or a pharmaceutically acceptable salt thereof and one or more other therapeutically active agents for use in the treatment of IPF.

In another embodiment, the invention provides a method of treating IPF comprising administering a safe and effective amount of a combination comprising a compound of formula (I) or a pharmaceutically acceptable salt thereof and one or more therapeutically active agents.

In a further embodiment, the invention provides a combination comprising a compound of formula (I) or a pharmaceutically acceptable salt thereof and one or more therapeutically active agents in the manufacture of a medicament for use in the treatment of IPF.

In another aspect, the invention provides a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more other therapeutically active agents for use in the treatment of IPF.

In another embodiment, the invention provides a method of treating IPF comprising administering a safe and effective amount of a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more therapeutically active agents.

In a further embodiment, the invention provides a combination comprising 2,4-difluoro-N-{2-(methyloxy)-5-[4-(4-pyridazinyl)-6-quinolinyl]-3-pyridinyl}benzenesulfonamide or a pharmaceutically acceptable salt thereof and one or more therapeutically active agents in the manufacture of a medicament for use in the treatment of IPF.

In a further aspect, the invention provides a combination comprising 2,4-difluoro-N-{2-(methyloxy)-5-[4-(4-pyridazinyl)-6-quinolinyl]-3-pyridinyl}benzenesulfonamide and one or more other therapeutically active agents for use in the treatment of IPF.

In another embodiment, the invention provides a method of treating IPF comprising administering a safe and effective amount of a combination comprising 2,4-difluoro-N-{2-(methyloxy)-5-[4-(4-pyridazinyl)-6-quinolinyl]-3-pyridinyl}benzenesulfonamide and one or more therapeutically active agents.

In a further embodiment, the invention provides a combination comprising 2,4-difluoro-N-{2-(methyloxy)-5-[4-(4-pyridazinyl)-6-quinolinyl]-3-pyridinyl}benzenesulfonamide and one or more therapeutically active agents in the manufacture of a medicament for use in the treatment of IPF.

One embodiment of the invention provides the use of combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of formula (I) or a pharmaceutically acceptable salt thereof and another therapeutically active agent for use in the treatment of a fibrotic disease.

In one embodiment, the invention provides a pharmaceutical composition comprising a combination of 2,4-difluoro-N-{2-(methyloxy)-5-[4-(4-pyridazinyl)-6-quinolinyl]-3-pyridinyl}benzenesulfonamide or a pharmaceutically acceptable salt thereof and another therapeutically active agent for use in the treatment of a fibrotic disease.

In another embodiment, the invention provides a pharmaceutical composition comprising a combination of 2,4-difluoro-N-{2-(methyloxy)-5-[4-(4-pyridazinyl)-6-quinolinyl]-3-pyridinyl}benzenesulfonamide and another therapeutically active agent for use in the treatment of a fibrotic disease.

In another embodiment, the invention provides a pharmaceutical composition comprising a combination of a compound of formula (I) or a pharmaceutically acceptable salt thereof and another therapeutically active agent for use in the treatment of IPF.

In another embodiment, the invention provides a pharmaceutical composition comprising a combination of 2,4-difluoro-N-{2-(methyloxy)-5-[4-(4-pyridazinyl)-6-quinolinyl]-3-pyridinyl}benzenesulfonamide or a pharmaceutically acceptable salt thereof and another therapeutically active agent for use in the treatment of IPF.

In a further embodiment, the invention provides a pharmaceutical composition comprising a combination of 2,4-difluoro-N-{2-(methyloxy)-5-[4-(4-pyridazinyl)-6-quinolinyl]-3-pyridinyl}benzenesulfonamide and another therapeutically active agent for use in the treatment of IPF.

### BIOLOGICAL DATA

### Example 1

Phosphorylation of AKT is widely accepted as an indication of PI3-kinase activity, and is utilised here to obtain an IC₅₀ for the effect of 2,4-difluoro-N-{2-(methyloxy)-5-[4-(4-pyridazinyl)-6-quinolinyl]-3-pyridinyl}benzenesulfonamide on primary human lung fibroblasts isolated from the IPF patients, in addition to macrophages isolated from IPF bronchoalveolar lavage (BALF).

Fibroblasts are seeded in 96 well plates at a density of 10,000 cells per well. Following 24 hours of serum starvation, fibroblasts are pre-incubated for 15 minutes in serum free buffer containing a range of concentrations of 2,4-difluoro-N-{2-(methyloxy)-5-[4-(4-pyridazinyl)-6-quinolinyl]-3-pyridinyl}benzenesulfonamide (50 μl volumes per well, comprising 1:10 dilutions of inhibitor ranging from [3×10⁻¹²M] to [3×10⁻⁷M] in serum free DMEM). Fibroblasts are stimulated by the addition of foetal calf serum (FCS) to a final volume of 10%. 30 minutes after stimulation, supernatants are rapidly decanted, and the cell plate placed on ice. 35 μl of 1× Complete Lysis buffer is added to each well and incubated for 10 minutes at 4° C., and stored at −80° C. prior to further analysis. Following thawing on ice, cell lysates are transferred onto Meso Scale Discovery (MSD) capture plates containing electrodes pre-coated with capture antibodies directed against total and phospho-AKT (Ser 473). The assay protocol is completed as per manufacturer's instructions and plates analysed on an MSD SECTOR Imager. Data are read out as the % of total AKT phosphorylated at position Ser473. Using these or similar conditions 2,4-difluoro-N-{2-(methyloxy)-5-[4-(4-pyridazinyl)-6-quinolinyl]-3 pyridinyl}benzenesulfonamide exhibits an IC₅₀ for inhibition of FCS induced AKT phosphorylation of 2.58 nM (95% CI 0.83-8 nM) (expressed as geometric mean and 95% CI for fibroblasts isolated from n=4 IPF lungs, n=2 experiments per cell line).

In similar experiments, inhibition of AKT phosphorylation by 2,4-difluoro-N-{2-(methyloxy)-5-[4-(4-pyridazinyl)-6-quinolinyl]-3 pyridinyl}benzenesulfonamide is assessed in cells isolated from IPF BALF. Briefly, BAL cells are resuspended in 0.1% BSA RPMI media to a cell provide a cell count of 5.7×10⁴-8.3×10⁴ cells per well. BAL cells are added to a 96 well plate and incubated with a range of 2,4-difluoro-N-{2-(methyloxy)-5-[4-(4-pyridazinyl)-6-quinolinyl]-3 pyridinyl}benzenesulfonamide (0.1% DMSO and 0.1% BSA final concentration in the assay). After 25 minutes incubation at 37° C., 5% CO₂ the plate is spun for 5 min at 1600 rpm. The supernatants are decanted and 40 μl of phosphosafe lysis buffer containing protease inhibitors is added to each well. The plate is then snap frozen in liquid nitrogen and stored at −80° C. prior to further analysis. Following thawing on ice, cell lysates are transferred onto phospho Akt, total Akt Meso Scale Discovery (MSD) capture plates and processed as described above. Using these or similar conditions 2,4-difluoro-N-{2-(methyloxy)-5-[4-(4-pyridazinyl)-6-quinolinyl]-3 pyridinyl}benzenesulfonamide exhibits an IC₅₀ for inhibition of AKT phosphorylation of 0.54 nM (95% CI 0.28-1.08 nM) (expressed as geometric mean and 95% CI for macrophages isolated from n=3 IPF BALFs).

### Example 2

### Experimental Preparations and Protocols

### (a) Primary Human Cell Isolation

Primary human fibroblasts were isolated by explant culture from non-IPF lung tissue, obtained post mortem following sudden trauma, and IPF lung tissue obtained from the lungs of IPF patients which were removed for transplantation or biopsy. Explants were cultured in DMEM containing high glucose and sodium pyruvate (PAA laboratories, catalogue #E15-011) with penicillin/streptomycin (Sigma #P4333), amphotericin B (Sigma #A2942), L-glutamine (Gibco #25030) and 20% FBS (foetal bovine serum, Lonza, catalogue #14-801F, Lot #1SB003) at 37° C., 100% humidity, 10% CO₂. Cells were designated non-IPF (UCL marked 0110 and 0610) and IPF (UCL marked 0207, 0208, 0308, 0507 and 0508) based on medical diagnosis.

### (b) Primary Human Cell Culture

Human lung primary fibroblast cell lines were cultured in DMEM containing high glucose and sodium pyruvate supplemented with L-glutamine, penicillin/streptomycin and 10% FBS (complete DMEM) at 37° C., 100% humidity, 10% CO₂. Cells were split into T175 flasks (NUNC #159910) 2 to 3 days prior to assay set-up at a density which yields approximately 70-80% confluence at time of harvest for assay. Cells were harvested using 0.25% trypsin-EDTA (Gibco #25300), washed and re-suspended in complete DMEM. Cell counts were performed on the cell suspension using a Handheld Automated Cell Counter (Millipore #PHCC00000) with 60 μm sensors (Millipore #PHCC60050).

(c) Measurement of pAKT

### Cell Culture

Primary fibroblasts were seeded into 96 well flat bottom plates (Nunc, #167008) at 10,000 cells per well in 100 μL of complete DMEM. Cells were not seeded in the outer wells, which were filled with 200 μL complete DMEM alone. Cells were incubated at 37° C. with 10%, 100% humidity, CO₂ for approximately 16-20 hours. Media on cells was changed to DMEM without FBS and incubated at 37° C. with 5% CO₂ for approximately 24 hours.

2,4-Difluoro-N-{2-(methyloxy)-5-[4-(4-pyridazinyl)-6-quinolinyl]-3 pyridinyl}benzenesulfonamide was serially diluted by a factor of 10 in DMSO (Sigma #D2650) 6 times from a starting concentration of 0.3 mM (stock concentration 30 mM was serially diluted 1:10 twice in DMSO to make the starting concentration). 2,4-Difluoro-N-{2-(methyloxy)-5-[4-(4-pyridazinyl)-6-quinolinyl]-3 pyridinyl}benzenesulfonamide was then further diluted 1:1000 into DMEM. Media was removed from the cells and 100 μL of diluted compound was added to each well (replicates of 6 wells, media plus 0.1% DMSO was added to both sets of control wells). Plates were incubated at 37° C. with 10% CO₂ for 15 min. Media was removed and replaced with DMEM plus 20% FBS containing the same dilution series of 2,4-difluoro-N-{2-(methyloxy)-5-[4-(4-pyridazinyl)-6-quinolinyl]-3 pyridinyl}benzenesulfonamide. Six control wells were replaced with DMEM plus 0.1% DMSO (negative control) and 6 with DMEM, 0.1% DMSO plus 20% FBS (positive control). Plates were incubated at 37° C., 100% humidity, 10% CO₂ for 30 minutes. Media was removed, plates placed on ice and 35 μL of ice cold Meso-Scale Discovery (MSD) lysis buffer (plus inhibitors as per MSD standard protocol) was added to each well. Plates were then immediately frozen at −80° C.

### MSD Assay

Meso-Scale Discovery (MSD) Phospho (Ser473)/Total Akt Whole Cell Lysate Kit (MSD #K15100D-2) were pre-blocked for 1 hour by adding 150 μL of 3% Blocker A solution. Plates were washed 4 times with 300 μL 1×MSD wash buffer (50 mM Tris pH 7.5, 150 mM NaCl, 0.02% Tween-20). The plate containing the cell lysates were defrosted on ice and 25 μL of lysate transferred to the prewashed AKT duplex plate. Plates were sealed and incubated at room temperature for 90 minutes, while shaking at 200 rpm.

AKT duplex plates were aspirated and washed 4 times with 300 μL 1×MSD Wash Buffer. After the final wash was aspirated, 25 μL of Detection Buffer (with 1× detection antibody) was added to each well. Plates were sealed and incubated for 1 hour while shaking at room temperature as above.

AKT duplex plates were then aspirated and washed 4 times with 300 μL 1×MSD Wash Buffer. After the final wash was aspirated, 150 μL of 1× Read Buffer (4× Read Buffer diluted in double distilled H₂O) was added to each well. Plates were read on a SECTOR™ Imager 6000 using MSD Workbench software.

### (d) Measurement of Population Cell Growth

### Cell Culture

Primary fibroblasts were seeded into 96 well flat bottom plates at 2,500 cells per well in 100 μL of complete DMEM. Cells were not seeded in the outer wells, which were filled with 200 μL complete DMEM alone. Cells were incubated at 37° C., 100% humidity, 10% CO₂ for approximately 16-20 hours. Media on cells was changed to DMEM without FBS and incubated at 37° C., 100% humidity, 10% CO₂ for approximately 24 hours.

2,4-Difluoro-N-{2-(methyloxy)-5-[4-(4-pyridazinyl)-6-quinolinyl]-3 pyridinyl}benzenesulfonamide was serially diluted by a factor of 10 in DMSO 8 times from a starting concentration of 30 mM. 2,4-Difluoro-N-{2-(methyloxy)-5-[4-(4-pyridazinyl)-6-quinolinyl]-3 pyridinyl}benzenesulfonamide was then further diluted 1:1000 in DMEM plus 10% FBS. Media was removed from the cells and 100 μL compound added to 6 wells per dilution. An additional 6 wells received media plus 10% FBS and 0.1% DMSO and 6 wells received media plus 0.1% DMSO. Wells A2-6 had media plus 0.1% DMSO added and A7-11 had media plus 10% FBS and 0.1% DMSO (no cell controls). Plates were incubated at 37° C., 100% humidity, 10% CO₂ for 72 hours. The T0 (time zero) control plate had 6 wells with media without FBS and 6 wells media plus 10% FBS as well as the same no cell controls mentioned previously.

### MTS Assay

For reading 20 μL of CellTiter 96™ AQueous non-radioactive cell growth assay (MTS) reagent (Promega #G5430) was added to all wells. Plates were incubated for a further 2 hours at 37° C., 100% humidity, 10% CO₂ then read at 490 nm on a Versaₘₐₓ microplate reader using Softmax Pro v5 software. Readings were corrected for FBS discoloration as per manufacturer's instructions, by the subtraction of values from media control (no cell) wells.

### (e) Measurement of Pro-Collagen Accumulation

### Cell Culture Conditions

Cultured non-IPF cell lines (as described in section 3.1.2) were used to study pro-collagen accumulation in cell culture supernatants, following TGF6 induced myofibroblast differentiation.

Cells were seeded at 100,000 cells per well in 12-well plates (NUNC #150628) in 1 mL complete DMEM and incubated until they reached 100% confluence (4-5 days). Medium was removed from the confluent cells and replaced with 1 mL pre-incubation medium (DMEM containing 4 mM glutamine, 50 μg/mL ascorbic acid, 0.2 mM proline and 0.4% FBS) and incubated for a further 24 hours. Fresh pre-incubation medium was added to 3 wells and immediately collected for analysis in order to determine background level (T0) of hydroxyproline present in the culture medium at the start of the incubation period.

TGFβ induces a slow activation of pAKT which peaks at 24 hours post TGFβ addition (data not shown). This suggests AKT activation is maximal subsequent to TGFβ driven myofibroblast differentiation (which usually occurs at 18-24 hours; data not shown. In order to investigate the effects of 2,4-difluoro-N-{2-(methyloxy)-5-[4-(4-pyridazinyl)-6-quinolinyl]-3 pyridinyl}benzenesulfonamide on PI3K activation in differentiated myofibroblasts, fibroblasts were initially differentiated with TGFβ for 24 hours prior to addition of 2,4-difluoro-N-{2-(methyloxy)-5-[4-(4-pyridazinyl)-6-quinolinyl]-3 pyridinyl}benzenesulfonamide. The effect of 2,4-difluoro-N-{2-(methyloxy)-5-[4-(4-pyridazinyl)-6-quinolinyl]-3 pyridinyl}benzenesulfonamide on pro-collagen production was assessed in the absence for a further 24 or 48 hours.

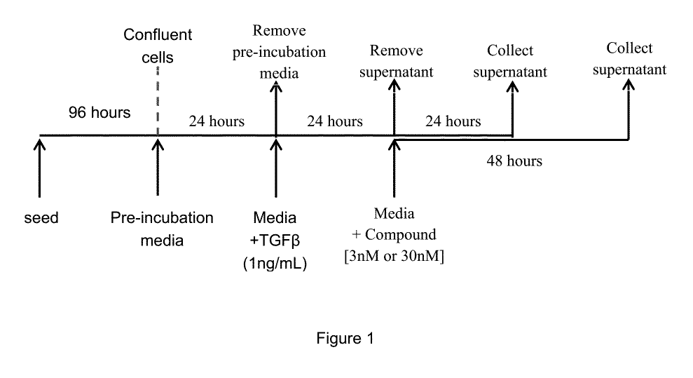

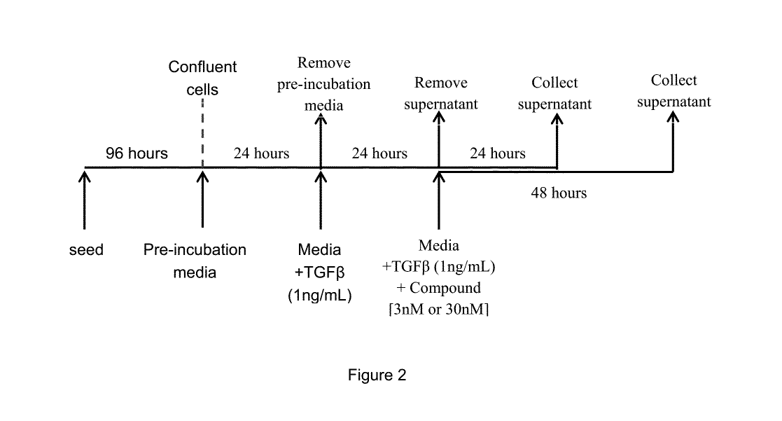

Following 24 hours serum starvation, fibroblasts were stimulated with TGFβ1 (1 ng/mL, 24 hours) (R&D #101-B1) to induce myofibroblast differentiation. Subsequently TGFβ1 was either removed or replenished (by complete serum free media change±TGFβ, 1 ng/ml) and 2,4-difluoro-N-{2-(methyloxy)-5-[4-(4-pyridazinyl)-6-quinolinyl]-3 pyridinyl}benzenesulfonamide (final concentration 3 nM or 30 nM, in 0.1% DMSO) added. Dilutions of 2,4-difluoro-N-{2-(methyloxy)-5-[4-(4-pyridazinyl)-6-quinolinyl]-3 pyridinyl}benzenesulfonamide were prepared from a stock of 0.03M by serial 10-fold dilution in 0.1% DMSO and a further 1000-fold dilution. Differentiated myofibroblast cultures were incubated for further 24 and 48 hours at 37° C., 100% humidity, 10% CO₂. See FIG. 1 and FIG. 2 for schematic of the experimental regimen.

### Cell Harvesting

At the end of each incubation time point cell supernatant was collected and frozen at −80° C. The cell layer was detached by incubating with 250 μL of 0.25% trypsin-EDTA for 5 minutes and a 1 mL suspension was made in DMEM. Cell counts were performed on the cell suspension using Sceptre 60 μM sensor.

The cell supernatants were allowed to thaw at room temperature and proteins were precipitated overnight in 67% (v/v) ethanol at 4° C.

### Recovery in Ethanol Insoluble Fraction

Precipitated proteins were recovered by vacuum filtration onto Durapore polyvinylidene difluoride (PVDF) membrane filters (pore size 0.45 μm) (type HV, Millipore Ltd., UK; # HVLP02500) and the adhering protein was washed twice with 1.5 mL ethanol (67% v/v). Filters with adherent protein were transferred to Pyrex hydrolysis tubes containing 2 mL 6M HCl. Ethanol-insoluble fractions were then hydrolysed at 110° C. for 16 h and the samples were decolourised by mixing with approx. 70 mg of charcoal and filtered onto Durapore membrane filters (pore size 0.65 μm) (type DA, Millipore Ltd., UK; DVPP02500). Aliquots (100 μl) of decolourised hydrolysate were transferred to 1.5 mL centrifuge tubes and evaporated to dryness using speed vac concentrator (Savant SPD 131DDA, Thermo Electron Corporation, Cambridge, UK).

### Derivitising Samples

Hydroxyproline accumulation in cell culture supernatants is used as a measure of pro-collagen production. Hydroxyproline represents approximately 12% of the primary sequence of pro-collagen and is essential for the formation of the collagen triple helix. Hydroxproline is not present in significant levels in any other proteins. Levels of hydroxyproline in cell culture hydrolysates was quantified by reverse-phase high performance liquid chromatography (HPLC) following derivitisation with 7-chloro-4-nitrobenzo-oxa-1; 3-diazole (NBD-CI) (Sigma; #17239-0050).

Hydroxyproline Standard: Standard samples of Trans-4-hydroxy-L-proline (PHPRO) Sigma; #H5534) are stored frozen at −20° C. in 10 μL (250 μM) aliquots. A 10 μL aliquot was diluted in 990 μL Milli-Q water (Milli-Q Plus; Millipore Ltd., UK) and used as standard. The final amount of Hydroxyproline (Hyp) standard loaded onto the column was 50 pmoles.

Samples: Milli-Q water (100 μL) was added to the dried aliquot of hydrolysate and left to rehydrate at 4° C. overnight. To 100 μL of each standard and sample 100 μL 0.4M potassium tetra borate buffer (adjusted to pH9.5 with HCl) and 100 μL NBD-CI (36 mM in methanol) was added. These were Vortex mixed thoroughly and incubated at 37° C. (in the dark) for 20 minutes. The reaction was stopped by adding 50 μL 1.5M HCl and 150 μL of a concentrated solution (3.33×) of HPLC running Buffer A (5.68 g sodium acetate dissolved in 150 mL Milli-Q water and 65 mL acetonitrile, corrected to pH6.4 with orthophosphoric acid and made up to 250 mL) by Vortex mixing thoroughly. The reaction mixture was drawn up into 1 ml syringe and filtered through an HPLC low dead volume filter (pore size 0.22 μm, type GV; #611-0716 Millipore Ltd, UK) into a plastic insert. The insert was placed into a brown glass tube (Laboratory Sales Ltd., Rochdale, UK), covered with a cap and the air bubbles were released by flicking gently at the bottom. These vials were then placed in the automatic sampler in the HPLC apparatus (Beckman Coulter, UK) and the samples were sequentially injected onto the HPLC column and eluted with an acetonitrile gradient as described in Table 1.

TABLE 1
Conditions and Buffers for the Separation of Hydroxyproline by
Reverse-Phase HPLC

| | |
|---|---|
| Column | LiChrosopher, 100 RP-18, 250 × 4 mm, 5 μm |
| Mobile phase | Buffer A - aqueous acetonitrile (8% v/v) |
| | 50 mM sodium acetate, pH 6.4 |
| | Buffer B - aqueous acetonitrile (75% v/v) |
| Column flow rate | 1.0 ml/min |
| Column temperature | 40° C. |
| Detection wavelength | 495 nm |

| | Time (min) | % Buffer B |
|---|---|---|
| Elution gradient | 0 | 0 |
| | 5 | 5 |
| | 6 | 80 |
| | 12 | 80 |
| | 12.5 | 0 |
| | 25 | 0 |

### Data Analysis

### (a) Concentration Response Curves for 2,4-difluoro-N-{2-(methyloxy)-5-[4-(4-pyridazinyl)-6-quinolinyl]-3-pyridinyl}benzenesulfonamide: pAKT response

For analysis of pAKT response, raw data from the sector imager was calculated to give the ratio of total AKT which was phosphorylated. Ratio values in the presence of increasing log Molar concentrations of 2,4-difluoro-N-{2-(methyloxy)-5-[4-(4-pyridazinyl)-6-quinolinyl]-3-pyridinyl}benzenesulfonamide were obtained. A mean and SEM of triplicate wells was obtained for each compound concentration (or no compound control) in each experiment. Data were expressed as a % of the maximum value obtained in the presence of 20% FCS without compound (% FCS control). Non-linear regression curves were fitted using a 4 parameter curve fit (Prism) was used to calculate IC₅₀ for each experiment.

### (b) Concentration Response Curves for 2,4-difluoro-N-{2-(methyloxy)-5-[4-(4-pyridazinyl)-6-quinolinyl]-3-pyridinyl}benzenesulfonamide: MTS Assay

The maximum fitted (upper asymptote) value for each experiment and used as the 100% reference response. The MTS signal (with media background values subtracted) in the presence of increasing log Molar concentrations of 2,4-difluoro-N-{2-(methyloxy)-5-[4-(4-pyridazinyl)-6-quinolinyl]-3-pyridinyl}benzenesulfonamide were expressed as a % of the maximum asymptote. No compound/FBS control values were also plotted. A mean and SEM of 5 or 6 replicates was obtained for each compound concentration (or no compound control) in each experiment. Non-linear regression curves were fitted using a 4 parameter curve fit (Prism) and used to calculate IC₅₀ for each cell line.

In a subset of experiments, where cell growth was measured in parallel with activation of caspase 3/7, T0 readings were taken when serum free medium was changed to 10% FBS and compound added. MTS values which dropped below T0 following 72 hours incubation with compound were assumed to represent cell death. For this experimental series values are normalized to T0 as 100% for the generation of IC₅₀ curves.

### (c) Effect of 2,4-difluoro-N-{2-(methyloxy)-5-[4-(4-pyridazinyl)-6-quinolinyl]-3-pyridinyl}benzenesulfonamide on Pro-Collagen Accumulation in Myofibroblast Supernatants

Quantification of the hydroxyproline (Hyp) content in each 100 μl sample injected into the column was determined by comparing peak areas of chromatograms obtained for each sample to those generated from the standard solutions derivatised and separated under identical conditions at the beginning and end of each experiment. The Hyp standard solutions derivatised were equivalent to 50 pmol/L Hyp and were used for calibration. Total collagen was normalised to cell number/mL and expressed as μg/cell and derived from the equation:

Total collagen=(sample peak area/standard peak×50)*×20**×8.1967***×131.135****/normaliser cell number.

### Derived Correction Factors:

*=Calculation for pmol of hydroxyproline on the column (normalised against a 50 pmol standard)
**=Correction for pmol hydroxyproline per well ( 1/20^{th} v/v of total well hydroxyproline was injected onto the column)
***Correction factor for the proportion of collagen accounted for by hydroxyproline.
****Molecular weight of hydroxyproline.

Data represent the mean±SEM of values obtained in groups of three wells per treatment. Statistical evaluation was performed using 2-Way ANOVA for group comparisons. A p value less than 0.05 was considered significant.

The T0 value represents the hydroxyproline present in the culture medium at the start of the incubation period.

### Results

### (i) Effect of 2,4-difluoro-N-{2-(methyloxy)-5-[4-(4-pyridazinyl)-6-quinolinyl]-3-pyridinyl}benzenesulfonamide on AKT phosphorylation

Intracellular inhibition of PI3K activity was determined by measuring the inhibition of AKT phosphorylation (pAKT) at position Ser473. The extent of AKT phosphorylation is an indirect measure of PI3K activity. PIP₃, the product of PI3K activation, is required for the localization of AKT to the plasma membrane, where upon it is phosphorylated by 3-phosphoinositide-dependent kinase-1 (PDK1) at site T308 and site S473 by the TORC2 (target of rapamycin complex 2).

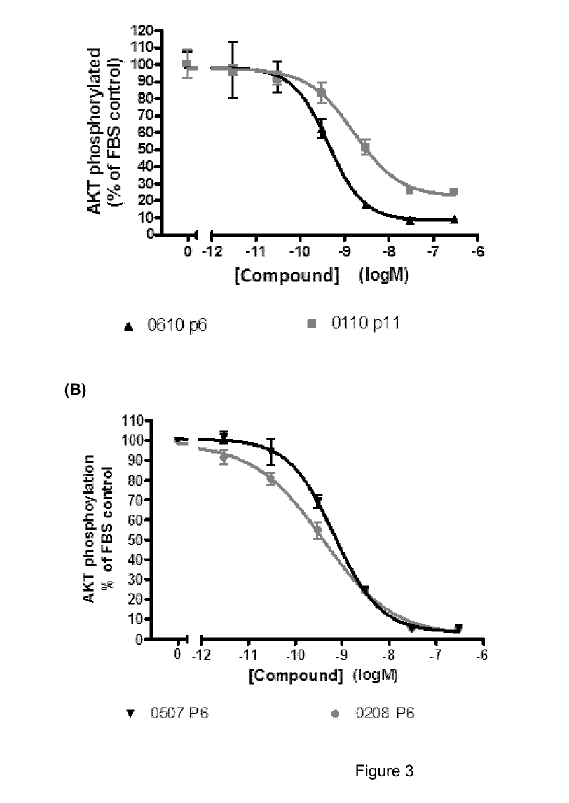

Using a Meso Scale Discovery (MSD) 96 well plate assay, the phosphorylation of AKT (S473) was measured in the presence of 2,4-difluoro-N-{2-(methyloxy)-5-[4-(4-pyridazinyl)-6-quinolinyl]-3-pyridinyl}benzenesulfonamide following a 30 minute stimulation by 20% FBS. The addition of 20% FBS caused an increase in AKT phosphorylation in all lines tested. FIG. 3 shows that AKT phosphorylation decreased in a concentration dependent manner following incubation with 2,4-difluoro-N-{2-(methyloxy)-5-[4-(4-pyridazinyl)-6-quinolinyl]-3-pyridinyl}benzenesulfonamide. Calculated mean pIC₅₀s were 9.1 (range 8.8-9.4, n=2 fibroblast lines, n=1 replicate passages per line) in primary human non-IPF (FIG. 3 (A)) and 8.8 (+/−0.1 SEM, n=5 cell lines, up to n=4 replicate passages per line) in IPF fibroblasts (FIG. 3 (B)).

### (ii) Effect on cell population growth of 2,4-difluoro-N-{2-(methyloxy)-5-[4-(4-pyridazinyl)-6-quinolinyl]-3-pyridinyl}benzenesulfonamide (MTS Assay)

The potency of 2,4-difluoro-N-{2-(methyloxy)-5-[4-(4-pyridazinyl)-6-quinolinyl]-3-pyridinyl}benzenesulfonamide for inhibition of cell growth was determined by a standardized 72 hour assay using CellTiter 96™ AQueous non-radioactive cell growth assay that quantifies mitochondrial activity as a surrogate of cell number.

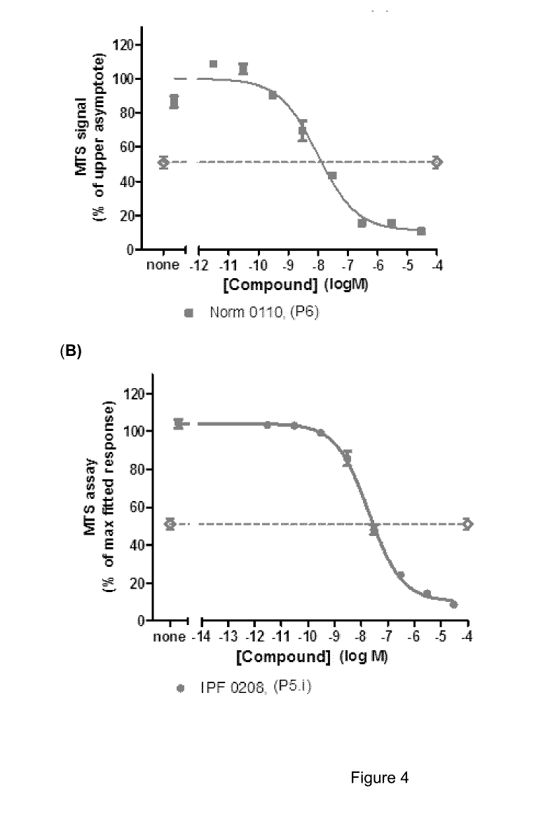

Exposure of fibroblasts to 10% FBS increased cell growth over 72 hours. Data presented in FIG. 4 (A & B) show growth of primary human lung fibroblasts was reduced in a concentration dependant manner following incubation with 2,4-difluoro-N-{2-(methyloxy)-5-[4-(4-pyridazinyl)-6-quinolinyl]-3-pyridinyl}benzenesulfonamide.

Calculated mean pIC₅₀s in were 7.7 (±0.2 SEM n=2 fibroblast lines, n=5 replicate passages per line) in primary human non-IPF (FIG. 4 (A)) and 7.5 (+/−0.1 SEM, n=5 cell lines, up to n=5 replicate passages per line) in IPF fibroblasts. At concentrations of 2,4-difluoro-N-{2-(methyloxy)-5-[4-(4-pyridazinyl)-6-quinolinyl]-3-pyridinyl}benzenesulfonamide >30 nM, the MTS signal for fibroblasts in the presence of 10% FBS appeared lower than no FBS controls. This suggests that 2,4-difluoro-N-{2-(methyloxy)-5-[4-(4-pyridazinyl)-6-quinolinyl]-3-pyridinyl}benzenesulfonamide was inducing a reduction in cell number at higher concentrations, potentially indicative of cell death.

### (iii) Effect of 2,4-difluoro-N-{2-(methyloxy)-5-[4-(4-pyridazinyl)-6-quinolinyl]-3-pyridinyl}benzenesulfonamide on pro-collagen synthesis

Pro-collagen production was assessed in primary human lung fibroblasts differentiated into myofibroblasts by measuring the accumulation of hydroxyproline in the culture supernatant, following TGFβ induced differentiation.

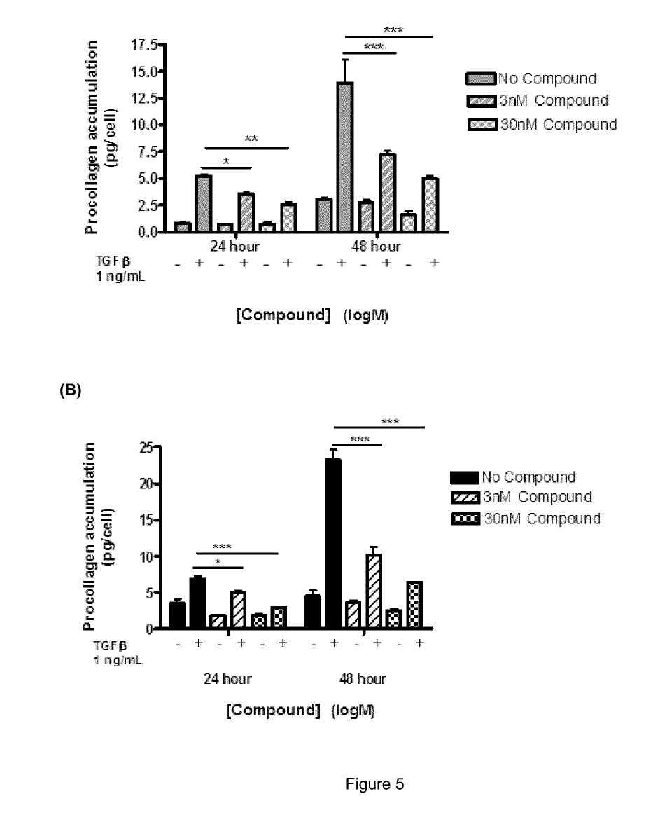

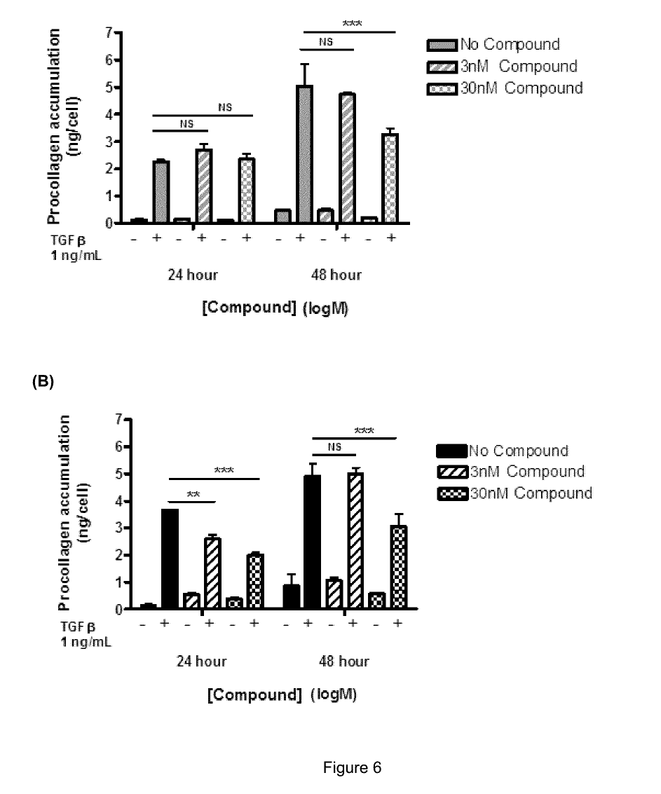

2,4-Difluoro-N-{2-(methyloxy)-5-[4-(4-pyridazinyl)-6-quinolinyl]-3-pyridinyl}benzenesulfonamide significantly reduced levels of pro-collagen produced by primary human lung fibroblasts, following TGFβ (1 ng/ml) induced differentiation into myofibroblasts (FIG. 5 & FIG. 6). The inhibitory effect was most marked when TGFβ was removed from culture media following myofibroblast differentiation (FIG. 5 (A & B)). In these experiments, pro-collagen accumulation persists despite the removal of TGFβ, for up to 48 hours. The accumulation of pro-collagen was sensitive to incubation with 2,4-difluoro-N-{2-(methyloxy)-5-[4-(4-pyridazinyl)-6-quinolinyl]-3-pyridinyl}benzenesulfonamide (3 nM and 30 nM, 24 and 48 hours, n=2 fibroblast lines). Mean percentage inhibition of pro-collagen accumulation by 2,4-difluoro-N-{2-(methyloxy)-5-[4-(4-pyridazinyl)-6-quinolinyl]-3-pyridinyl}benzenesulfonamide is summarised for 2 fibroblast lines in Table 2 (A & B).

TABLE 2
Summary of values for mean percent inhibition of TGFβ
induced pro-collagen production by 2,4-difluoro-N-{2-
(methyloxy)-5-[4-(4-pyridazinyl)-6-quinolinyl]-3-
pyridinyl}benzenesulfonamide are shown for lines 0110
(A) and 0610 (B)

[Compound] (nM)

| | |
|---|---|
| 3 nM | 30 nM |

(A)

| | | |
|---|---|---|
| 24 hour | 31.61% | 60.80% |
| 48 hour | 48.06% | 63.95% |

(B)

| | | |
|---|---|---|
| 24 hour | 26.25% | 56.82% |
| 48 hour | 35.63% | 59.61% |

Percentages are calculated by comparing compound treated values to no compound controls for n = 3 wells.

In experiments where TGFβ was replenished into the incubation media at the same time as compound, inhibition of pro-collagen production was less marked (FIG. 6 (A & B)). Mean percentage inhibition of pro-collagen accumulation by 2,4-difluoro-N-{2-(methyloxy)-5-[4-(4-pyridazinyl)-6-quinolinyl]-3-pyridinyl}benzenesulfonamide is summarised for 2 fibroblast lines in Table 3 (A & B).

TABLE 3
Summary of values for mean percent inhibition of TGFβ
induced pro-collagen production by 2,4-difluoro-N-{2-
(methyloxy)-5-[4-(4-pyridazinyl)-6-quinolinyl]-3-
pyridinyl}benzenesulfonamide are shown for lines 0110
(A) and 0610 (B) following replenishment of TGFβ

[Compound] (nM)

| | |
|---|---|
| 3 nM | 30 nM |

(A)

| | | |
|---|---|---|
| 24 hour | No inhibition | No inhibition |
| 48 hour | 6.3% | 35.22% |

(B)

| | | |
|---|---|---|
| 24 hour | 29.15% | 45.40% |
| 48 hour | No inhibition | 37.68% |

Percentages are calculated by comparing compound treated values to no compound controls for n = 3 wells.

### DISCUSSION

2,4-Difluoro-N-{2-(methyloxy)-5-[4-(4-pyridazinyl)-6-quinolinyl]-3-pyridinyl}benzenesulfonamide decreased the level of phospho-AKT, a downstream target of PIP3, in a concentration dependent manner. The proliferation of fibroblasts was also reduced by increasing levels of 2,4-difluoro-N-{2-(methyloxy)-5-[4-(4-pyridazinyl)-6-quinolinyl]-3-pyridinyl}benzenesulfonamide. Both these results suggest that 2,4-difluoro-N-{2-(methyloxy)-5-[4-(4-pyridazinyl)-6-quinolinyl]-3-pyridinyl}benzenesulfonamide has the potential to reduce fibroblast proliferation and increase fibroblast apoptosis at concentrations >300 nM. Analysis of collagen levels suggests that 2,4-difluoro-N-{2-(methyloxy)-5-[4-(4-pyridazinyl)-6-quinolinyl]-3-pyridinyl}benzenesulfonamide prevents TGF-β induced collagen production, in cells which have been driven to differentiate into myofibroblasts by TGFβ stimulation.

Taken together these data show that 2,4-difluoro-N-{2-(methyloxy)-5-[4-(4-pyridazinyl)-6-quinolinyl]-3-pyridinyl}benzenesulfonamide potently inhibits mechanisms important in the progression of IPF namely fibroblast proliferation and collagen production.

What is claimed is:

1. A method of treating idiopathic pulmonary fibrosis comprising administering a safe and effective amount of a compound which is 2,4-difluoro-N-{2-(methyloxy)-5-[4-(4-pyridazinyl)-6-quinolinyl]-3-pyridinyl}benzenesulfonamide, represented by the structure: 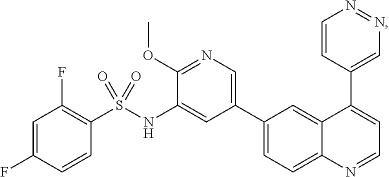 [SMILES: COc1ncc(-c2ccc3nccc(-c4ccnnc4)c3c2)cc1NS(=O)(=O)c1ccc(F)cc1F]
or a pharmaceutically acceptable salt thereof to a patient in need thereof.

2. A method of treating idiopathic pulmonary fibrosis comprising administering a safe and effective amount of a compound which is 2,4-difluoro-N-{2-(methyloxy)-5-[4-(4-pyridazinyl)-6-quinolinyl]-3-pyridinyl}benzenesulfonamide, represented by the structure: 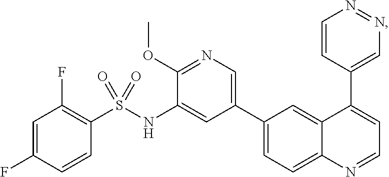 [SMILES: COc1ncc(-c2ccc3nccc(-c4ccnnc4)c3c2)cc1NS(=O)(=O)c1ccc(F)cc1F]
to a patient in need thereof.
